(19) United States
(12) Patent Application Publication
(10) Pub. No.: US 2022/0205881 A1
(43) Pub. Date: Jun. 30, 2022

(54) METHOD FOR MICROSCOPIC EXAMINATION OF PHYTOPLANKTON

(71) Applicant: Northeast Institute of Geography and Agroecology, Chinese Academy of Sciences, Changchun City (CN)

(72) Inventor: Xiaonan LOU, Changchun City (CN)

(73) Assignee: Northeast Institute of Geography and Agroecology, Chinese Academy of Sciences, Changchun City (CN)

(21) Appl. No.: 17/508,444

(22) Filed: Oct. 22, 2021

(30) Foreign Application Priority Data
Dec. 28, 2020 (CN) 202011576372.0

(51) Int. Cl. G01N 1/28; G01N 15/10

(52) U.S. Cl. CPC G01N 1/28; G01N 33/1826; G01N 15/10

(57) ABSTRACT

The present disclosure relates to a method for microscopic examination of phytoplankton. The present disclosure solves the problem of decreased counting accuracy of perioptometry in a dry environment. The method is as follows: step 1, making an analyte into a glass slide, performing qualitative identification of the phytoplankton in the field of view; and step 2, drying the glass slide; after drying, counting the phytoplankton and full-slide rare algae on the glass slide microscopically. When the humidity of microscopy room is only 20%, the method of the present disclosure may count a sample accurately, which has a significant increase in counting accuracy compared with the existing perioptometry. The method of the present disclosure effectively solves the problem of failure to perform the perioptometry accurately due to dry ambient humidity.

### CROSS REFERENCE TO RELATED APPLICATION(S)

This patent application claims the benefit and priority of Chinese Patent Application No. 202011576372.0 filed on Dec. 28, 2020, the disclosure of which is incorporated by reference herein in its entirety as part of the present application.

### TECHNICAL FIELD

The present disclosure relates to a method for microscopic examination of algae.

### BACKGROUND ART

The main research method of phytoplankton in freshwater lakes and reservoirs is achieved by counting algae under a microscope. The conventional method for counting phytoplankton comprises taking 0.1 mL of sample and counting it in the field of a microscope after magnification through the 40× objective lens by 10× eyepiece of the microscope. Perioptometry is mostly used as the counting method. When perioptometry is used for counting, the ambient humidity of the microscopy room is required to be between 70% and 80%. However, excessively high ambient humidity limits the normal use of the microscope. The lens of the microscope is easy to mildew in the environment with high humidity for a long time. In the winter in North China, due to the dry environment caused by the climate, the humidity in the microscopy room is generally around 20%. In this extremely dry environment, because the water of the sample on the counting slide evaporates too rapidly and the algae in the sample on the evaporated glass slide will move as the water evaporates without completing the counting process, the process of the perioptometry will fail, and the accuracy of the counting process of the sample with higher biomass will decrease.

### SUMMARY

In order to solve the problem of decreased counting accuracy of perioptometry in a dry environment, the present disclosure provides a method for microscopic examination of phytoplankton.

The method for microscopic examination of phytoplankton provided by the present disclosure includes the following steps:

step 1, making an analyte into a glass slide, performing qualitative identification of the phytoplankton in the field of view, and determining dominant species, common species, and rare species of a sample to be counted; and

step 2, drying the glass slide in step 1; after drying, counting the phytoplankton and full-slide rare algae on the glass slide microscopically by using perioptometry, namely realizing the microscopic examination of the phytoplankton.

In the present disclosure, in the drying process, when drying a liquid sample is accelerated by external force, the glass slide is not allowed to face toward an air outlet position directly; furthermore, excessive wind must be avoided, and massive displacement of algae distributed on the glass slide is avoided due to external force, for fear of the aggregation of the algae on the glass slide.

In step 1 of the present disclosure, the qualitative identification of the phytoplankton algae is performed in the field of view to determine the dominant species, common species, and rare species of the sample to be counted; after a glass slide used for counting the sample is processed, the size and morphology of the algae change slightly. It is necessary to confirm the dominant species, common species, and rare species of the phytoplankton on the glass slide before counting. The operation in this step may ensure the accuracy in determining the type of processed samples to be counted.

In the present disclosure, the phytoplankton may be accurately counted when ambient humidity reaches 70%-80% in a microscopy room environment; when the humidity of microscopy room is only 20%, the method of the present disclosure may count a sample accurately, which has a significant increase in counting accuracy compared with the existing perioptometry. The method of the present disclosure effectively solves the problem of failure to perform the perioptometry accurately due to dry ambient humidity.

### DETAILED DESCRIPTION OF THE EMBODIMENTS

Embodiment 1: A method for microscopic examination of phytoplankton in this example includes the following steps:

step 1, making an analyte into a glass slide, performing qualitative identification of the phytoplankton in the field of view, and determining dominant species, common species, and rare species of a sample to be counted; and

step 2, drying the glass slide in step 1; after drying, counting the phytoplankton and full-slide rare algae on the glass slide microscopically, namely realizing the microscopic examination of the phytoplankton.

In the embodiment, in the drying process of step 2, as drying a liquid sample is accelerated by external force, the glass slide is not allowed to face toward an air outlet position directly; furthermore, excessive wind must be avoided, and massive displacement of the algae distributed on the glass slide is avoided due to external force, for fear of the aggregation of the algae on the glass slide.

Embodiment 2: The difference between this embodiment and Embodiment 1 is that: a dryer, a fan, or a blower is used in the drying in step 2. Other steps and parameters are the same as those in Embodiment 1.

In this embodiment, in the drying process, as drying a liquid sample is accelerated by external force, particularly by a fan or a blower, the glass slide is not allowed to face toward an air outlet position directly; furthermore, excessive wind must be avoided, and massive displacement of the algae distributed on the glass slide is avoided due to external force, for fear of the aggregation of the algae on the glass slide.

### EXAMPLE 1

The phytoplankton in samples of the freshwater reservoir Songhua Lake in August 2020 were counted by the perioptometry and the method of the present disclosure. Herein, the method for microscopic examination of phytoplankton provided by the present disclosure included the following steps:

step 1, an analyte was made into a glass slide, qualitative identification of the phytoplankton was performed in the field of view, and dominant species, common species, and rare species of a sample to be counted was determined; and

step 2, the glass slide in step 1 was dried in a dryer; after drying, the phytoplankton and full-slide rare algae were counted on the glass slide microscopically by using perioptometry.

The counting results were shown in Table 1. Herein, samples 1 and 2 were counted in a microscopy room with a humidity of 20%.

### EXPERIMENTAL COMPARISON RESULTS

| | | Present | | | Present |
|---|---|---|---|---|---|
| Perioptometry | | disclosure | Perioptometry | | disclosure |
| (algae) | Algae of sample 2 | (algae) | (algae) | Algae of sample 1 | (algae) |
| 162 | Fragilaria sp. | 604 | 290 | Fragilaria sp. | 632 |
| 14 | Synedra sp. | 35 | 17 | Synedra sp. | 57 |
| 0 | Stephanodiscus sp. | 2 | 0 | Stephanodiscus sp. | 1 |
| 0 | Cyclotella sp. | 8 | 2 | Cyclotella sp. | 1 |
| 0 | Melosira granulata (Ehr.) Ralfs | 30 | 16 | Melosira granulata (Ehr.) Ralfs | 1 |
| 14 | Microcystis sp. | 259 | 22 | Microcystis sp. | 21 |
| 0 | Raphidiopsis sp. | 48 | 24 | Aphanocapsa sp. | 4 |
| 0 | Scenedesmus arcuatus | 9 | 2 | Raphidiopsis sp. | 4 |
| 0 | Scenedesmus bijuga | 130 | 28 | Anabaena circinalis | 8 |
| 2 | Chlorella sp. | 4 | 0 | Ankistrodesmus sp. | 4 |
| 0 | Oocystis sp. | 16 | 4 | Chroomonas sp. | 2 |
| 0 | Ceratium hirundinella | 10 | 2 | Cyrptomonas ovata | 3 |
| 0 | Cosmarium sp. | 41 | 15 | Chlorella sp. | 2 |
| 1 | Chroomonas sp. | 8 | 0 | Scenedesmus bijuga | 4 |
| 0 | Cryptomonas ovata | 16 | 0 | Scenedesmus obliquus | 2 |

According to the experimental results in Table 1, when the humidity of the microscopy room is only 20%, the method of the present disclosure can accurately count the samples, which has an increase in counting accuracy compared with the existing perioptometry. The counting method of phytoplankton samples provided by the present disclosure effectively solves the problem of failure to perform the perioptometry accurately due to dry ambient humidity.

What is claimed is:

1. A method for microscopic examination of phytoplankton, comprising the following steps:
step 1, making an analyte into a glass slide, performing qualitative identification of the phytoplankton in the field of view, and determining dominant species, common species, and rare species of a sample to be counted; and
step 2, drying the glass slide in step 1; after drying, counting the phytoplankton and full-slide rare algae on the glass slide microscopically, namely realizing the microscopic examination of the phytoplankton.

2. The method according to claim 1, wherein the drying in step 2 is carried out by using a dryer, a fan, or a blower.
